(19) United States
(12) Patent Application Publication
(10) Pub. No.: US 2015/0173936 A1
(43) Pub. Date: Jun. 25, 2015

(54) UPPER EXTREMITY IMMOBILIZER

(71) Applicant: Richard M. Lowden, San Francisco, CA (US)

(72) Inventor: Richard M. Lowden, San Francisco, CA (US)

(21) Appl. No.: 14/571,650

(22) Filed: Dec. 16, 2014

Related U.S. Application Data
(60) Provisional application No. 61/918,566, filed on Dec. 19, 2013.

(51) Int. Cl. A61F 5/37

(52) U.S. Cl. CPC A61F 5/3753

(57) ABSTRACT

A shoulder immobilizer has a pair of resilient cushions in an L-configuration. A resilient elbow cushion is generally perpendicular to a body cushion that fits under the shoulder of a user. The elbow cushion provides rearward cushioning of the elbow, particularly in sleep positions of a user. A pair of tangent belts support the cushions. A torso belt surrounds a portion of a user's torso and is adhered to second or back sides of the two cushions. An elbow belt surrounds a portion of a user's elbow and is adhered to a second or back side of the elbow cushion at one end and fastened to the torso belt at an opposite end.

### CROSS-REFERENCE TO RELATED APPLICATION

This application claims priority from provisional patent application Ser. No. 61/918,566; filed Dec. 19, 2013.

### TECHNICAL FIELD

The invention relates to arm slings, and, in particular, to a sling that immobilizes the shoulder after a fracture or surgical procedure involving the shoulder.

### BACKGROUND ART

The shoulder comprises four main bones including the clavicle, the uppermost generally horizontal bone running towards the neck also known as the collarbone, the humerus, a generally downwardly extending bone associated with arm movement, the scapula, a generally downwardly extending bone associated with the torso and the acromion, an upward protrusion from the scapula extending at an angle as a partial pivot member against the clavicle. The joinder of these bones is the glenohumeral joint where the articulation between the top of the humerus and the lateral portion of the scapula occurs. Various soft tissue members form the actual joint. Breakage or fracture of any of these bones usually requires shoulder partial immobilization.

The problem with most immobilization devices is that they cause pain when pressure other than vertical is placed on the arm associated with the broken shoulder bone, such as by sleeping on one's side. Pain is frequently caused by backward extension of the arm while the arm is in abduction required for injury healing. While a small amount of backward arm rotation must be expected, the amount must be gently limited to avoid trauma. Other immobilization devices, such as shoulder slings, do not adequately limit movement when a person is horizontal and interfere with natural body movement in a significant manner, such as by restriction of a user's hand, or do not gently limit rearward arm rotation.

In U.S. Pat. No. 8,109,273 a shoulder immobilizer includes a semi-rigid or rigid orthosis, in the form of an arm support, which supports the upper arm, elbow, forearm and wrist of a patient. A bolster is positioned between the patient and the arm support. A body strap extends around the patient and attaches to the arm support and/or the bolster, holding the arm support and bolster in position against the body of the patient.

In U.S. Pat. No. 7,244,239 a shoulder stabilizing restraint includes a pillow and a forearm wrap. The pillow has an arcuately-configured posterior face for engaging an anterior quadrant of a torso on a first side of a user. The pillow also has a flat forearm engagement face for engaging a forearm on the first side of the user. The forearm engagement face preferably converges with the posterior face at a posterior/lateral convergence which is positionable proximal to a lateral border of the torso on the first side of the user. The forearm wrap is fastenable to the pillow to retain the forearm in engagement with the forearm engagement face and the shoulder joint at a positive external rotation angle.

In U.S. Pat. No. 7,244,239 a shoulder immobilizer includes apparatus for abduction of the upper limbs of a patient using a belt and at least one plate made of a rigid material which is capable of being deformed. A belt is provided to encompass the trunk of the patient and is equipped with at least one pocket which is intended to receive the crosswise segment of the plate. The vertical segment or segments of the plate are provided to hold the arm and the forearm of the patient in the desired position. The base of the deformed portion of the plate is supported.

While prior art devices are effective against gravity in a vertical orientation, they are bulky, cumbersome and often uncomfortable, as well as being ineffective in stabilizing the arm and shoulder when gravity acting on a horizontal body and arm, particularly while sleeping. What is needed and the object of the invention is a lightweight shoulder immobilizer with elbow support that stabilizes the arm when the person is in a horizontal position and this eliminates pain when a person is sleeping on her/his side (sidelying) or back (supine).

### SUMMARY OF INVENTION

The above object has been met with a new shoulder immobilizer that employs a pair of cushions of generally the same size that may be configured for use on either side of the user. The immobilizer features a resilient body cushion joined by a flexible hinge to a resilient elbow cushion where there is a junction of the cushions forming an L shaped configuration. A first belt contacts the body cushion and is adhered to the elbow cushion by Velcro. At the cushion junction the first belt wraps around the torso of user and is secured to a first fastener at a free end of the first belt forward of the body cushion. A second belt contacts the elbow cushion and is adhered to the elbow cushion by Velcro. The second belt has an end near the junction, then wraps around the elbow cushion and a human arm, then extends to a second fastener at the free end of the first belt. The two belts support the two cushions without shoulder support.

### BRIEF DESCRIPTION OF THE FIGURES

FIG. 1 is a front plan view of an upper extremity immobilizer, worn by a user, in accordance with the invention;

FIG. 2 is a side view thereof;

FIG. 3 is a top sectional view of the upper extremity immobilizer of FIG. 1.

### DESCRIPTION OF INVENTION

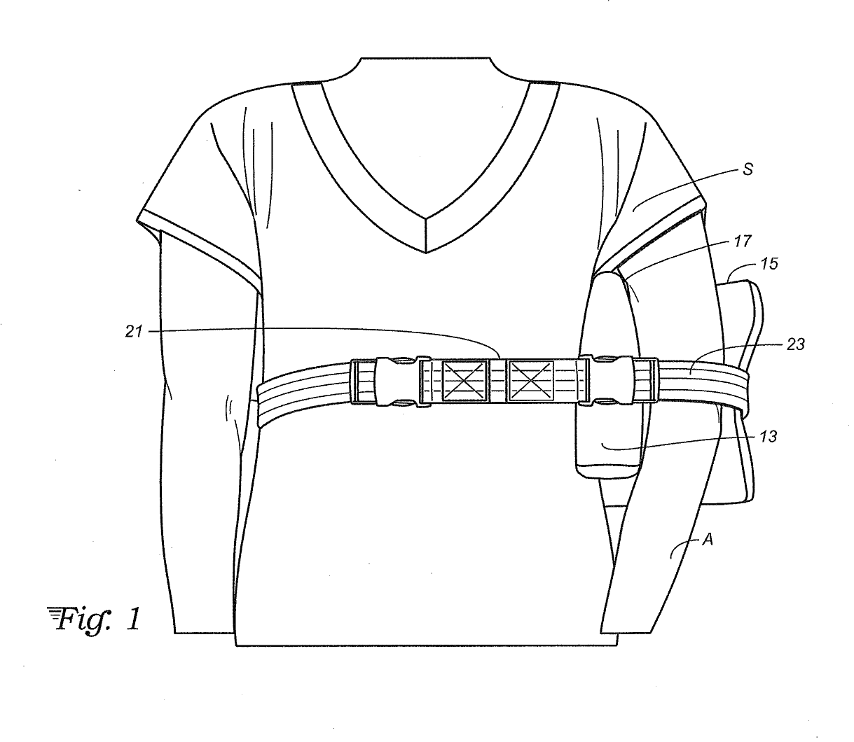

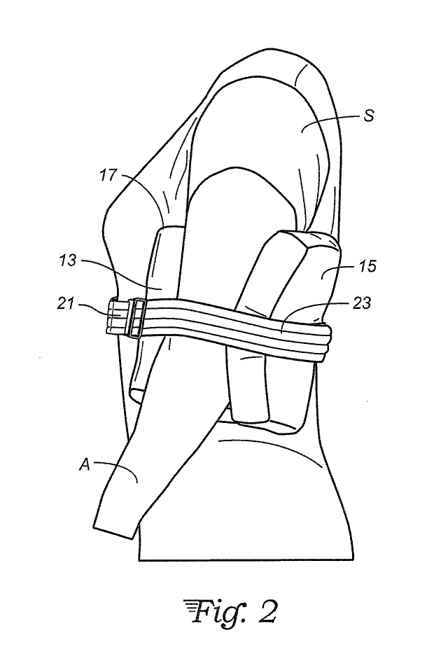

With reference to FIGS. 1 and 2, an upper extremity immobilizer is an L-shaped arrangement of a body cushion 13 that is upright under the shoulder, S, of a user and a generally perpendicular elbow cushion 15. The base of the L-shape is the elbow cushion 15 seen behind the upper part of the arm, A, of the user.

The upper part of the body cushion 13 is wedge shaped with the point 17 of the wedge extending towards the armpit, just below the shoulder, or towards the chest and the armpit. The body cushion is held in place by a torso belt 21 that surrounds a user's body at midsection or chest level, as described below. Wedge shaped cushions are generally known, as shown in U.S. Pat. No. 8,109,273.

The elbow cushion 15 works with body cushion 13 in the L-shaped configuration. The cushions are generally 5 inches by 8 inches. The body cushion is about 3.5 inches thick at its base and 1.5 inches thick at the apex. The elbow cushion is about 1.5 to 2 inches thick. The elbow cushion is a stop against rearward arm rotation and is behind the arm of a user as seen from the front. The '273 patent shows a cast type rigid member as a stop against rearward arm rotation, but such rigid members are known to cause pain or discomfort. We have found that a soft or resilient stop is effective against rearward arm rotation if there is a means to firmly position such a soft stop in place.

A resilient stop is formed by an elastomeric elbow cushion, such as could be formed by a woven fabric encasing a foam pad. The elbow cushion is held in place by an elbow belt 23 that attaches to the torso belt 21 by Velcro and is proximate to, or joined to, the body cushion, with each cushion having a surrounding belt at chest level. The belts are both closed and fastened at the front of the torso.

Both cushions have roughly the same areawise size and are made of similar material, such as woven fabric encasing foam of medium consistency that is yieldable but has shape retentive properties against forces that can be expected from a human arm. The woven fabric should adhere to Velcro and so the weave should not be tight but have the weave and texture of a fine woven outer summer garment. Together, the body cushion 13 and the elbow cushion 15, joined in an L-shaped configuration, form an effective immobilization apparatus for arms, allowing for shoulder bone and arm healing. That is particularly effective in sleep positions where rigid elbow stops cause discomfort leading to lack of sleep.

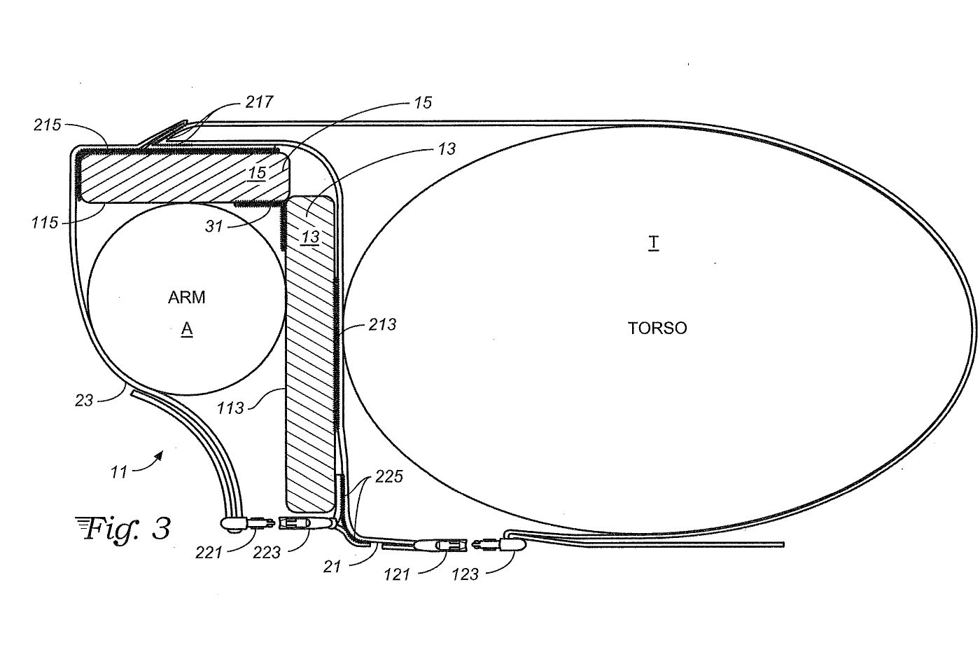

With reference to FIG. 3, elbow belt 23 is a belt loop that extends from the top of the L-shape, around an outward portion of arm A, and then around the second major surface 215 of the elbow cushion where it is fastened to the cushion by Velcro. The elbow cushion 15 is attached by a Velcro hinge 31, i.e. a short strap, to a first major surface 113 of body cushion 13 in an L-shaped configuration. The torso belt 21 wraps around a user's torso, T, with a front side of the torso belt 21 facing outwardly from torso T. A first pair of latch connectors 121, 123 allow fastening of the torso belt at the front of torso T. A portion of the front side of the belt 21 contacts a second major surface 213 of body cushion 13 and is held in place by Velcro near a location where a second pair of latch connectors 221, 223, are located with latch member 221 associated with an end of the elbow belt. The other latch member 223 is associated with the front side of the torso belt, being held in place by Velcro strip 225 at the forward end of body cushion 13.

The elbow belt 23 is like a smaller ring attached to a larger ring at a point of tangency. A second major surface 215 of elbow cushion 15 anchors a turning point 217 of torso belt 21 using Velcro. The turning point 217 may be anchored to an end region of elbow belt 23 where the elbow belt is anchored to the second major surface 215 of elbow cushion 15. The elbow belt 23 will allow some yielding against rearward arm rotation due to coupling to the torso belt. Rearward arm rotational force on the elbow cushion first compresses the elbow cushion, then works against the elbow belt, taking up any slack in the belt, then works against the torso belt, perhaps causing some rotation of the belt about the body of a user in the case of the strong rearward force against the elbow cushion. The amount of permitted rotation is adjusted by tightness of the belts. In combination, the L-shaped cushions, arranged in a generally perpendicular manner, and the two tangent belts offer an effective immobilization device, particularly for sleeping.

What is claimed is:

1. A shoulder immobilizer supported only at the waist of a user comprising:
a torso belt encircling the torso of a user with excess length supporting an L-shaped configuration of a body cushion and an elbow cushion from an outer surface of the belt;
an elbow belt joined to a first region of the torso belt at one end of the elbow belt near a forward portion of the L-shaped configuration of cushions, encircling the upper arm of a user, and at another end of the elbow belt joined to the torso belt at a second region;
wherein the body cushion and elbow cushion are foam filled cushions with the body cushion fitting between the arm and torso of a user in contact with the torso belt and the elbow cushion residing next to an arm of a user in perpendicular relation to the body cushion in contact with at least the elbow belt.

2. A shoulder immobilizer supported only at the waist of a user comprising:
a resilient rectangular body cushion having first and second opposed major surfaces terminating in a forward end and a rearward end and fitting beneath a shoulder joint of a user;
a resilient rectangular elbow cushion having first and second opposed major surfaces terminating in a junction end and a free end;
the first major surface of the body cushion joined by a hinge to the first major surface of the elbow cushion, the hinge having a first portion at the rearward end of the body cushion and a second portion at the junction end of the elbow cushion, the body cushion and the hinged elbow cushion forming an L-shaped configuration;
a torso belt contacting the second major surface of the body cushion toward the junction end of the elbow cushion then encircling a user's torso where ends of the belt are joinable; and
an elbow belt having an attachment region with a first end of the elbow belt attached to the torso belt near the junction end of the elbow cushion at an attachment region, with a length extending from the attachment region and surrounding the second major surface of the elbow cushion and a human arm, with a second end of the elbow belt joinable to an end of the torso belt.

3. The apparatus of claim 2 wherein opposed major surfaces of the body cushion are mutually vertically inclined forming a wedge shaped cushion having an apex region fitting below a human shoulder joint.

4. The apparatus of claim 3 wherein opposed major surfaces of the body cushion and the elbow cushion are woven material separated by a foam pad.

5. The apparatus of claim 4 wherein opposing major surfaces of the elbow cushion are substantially planar.

6. The apparatus of claim 2 wherein the hinge joining the first major surface of the body cushion and the first major surface of the elbow cushion is a strap having a strap surface of Velcro material facing said first surfaces and having separate Velcro regions hingedly joining said first major surfaces in the L-configuration.

7. The apparatus of claim 2 wherein a first Velcro belt strip is joined to the second major surface of the body cushion beneath the torso belt and joined thereto by Velcro material facing the torso belt.

8. The apparatus of claim 7 wherein the second major surface of the elbow cushion has a second Velcro strip facing the elbow belt and joined thereto by Velcro material facing the elbow belt.

9. The apparatus of claim 7 wherein the elbow belt has a third Velcro strip facing the torso belt at the attachment region.

10. The apparatus of claim 1 wherein the body cushion and the elbow cushion have generally the same areawise size.
